(19) United States
(12) Patent Application Publication
(10) Pub. No.: US 2010/0303916 A1
(43) Pub. Date: Dec. 2, 2010

(54) ENHANCED DRUG DELIVERY WITH ORIENTABLE PARTICLES

(75) Inventors: Warren H. Finlay, Edmonton (CA); Andrew R. Martin, Versailles (FR); Helen Orzanska, Edmonton (CA); Richard Thompson, Edmonton (CA)

Correspondence Address: Lambert Intellectual Property Law, Suite 200, 10328 - 81 Avenue, Edmonton, AB, T6E 1X2, CA

(73) Assignee: THE GOVERNORS OF THE UNIVERSITY OF ALBERTA, Edmonton, AB (CA)

(21) Appl. No.: 12/678,105

(22) PCT Filed: Sep. 23, 2008
(86) PCT No.: PCT/CA08/01672

§ 371 Date: Mar. 12, 2010

Related U.S. Application Data
(60) Provisional application No. 60/974,592, filed on Sep. 24, 2007.

(51) Int. Cl. A61K 9/14; A61K 31/353; A61P 11/00; A61M 37/00

(52) U.S. Cl. 424/489; 514/456; 604/500; 604/93.01

(57) ABSTRACT

Small airway deposition of orientable drug particles in the lung due to interception is increased through alignment of these particles with an externally applied force such as a magnetic field. Drug particles in one embodiment are made magnetically responsive by loading them with magnetic nanoparticles. Elongated particles have a natural tendency to align parallel to the direction of flow through an airway, and therefore also parallel to airway walls; accordingly, alignment with a magnetic field to any other orientation increases interception, with a maximum increase for alignment perpendicular to airway walls. By positioning a magnetic field across a specific site within the lung, for example in the area of a tumor, the increase in deposition by interception allows localized targeting of inhaled drug particles to that area.

### BACKGROUND

Respiratory tract deposition of inhaled pharmaceutical aerosols has been studied extensively due to its importance in determining drug dosages delivered to the lung. For compact, approximately spherical, aerosol particles, deposition primarily occurs through inertial impaction, gravitational sedimentation, or Brownian diffusion onto airway walls. In a given region of the respiratory tract, the probability of deposition due to these mechanisms is dependant on particle size and density, as well as on the airway geometry and flow patterns within that region.

In considering the inhalation of elongated, high aspect ratio particles, an additional deposition mechanism must be taken into account, this being the interception of the tip of a particle with an airway wall. Deposition by interception depends on the ratio between particle length and airway diameter, as well as on the orientation of a particle as it travels through an airway. Given that airway diameters decrease by almost two orders of magnitude between the trachea and the peripheral, gas-exchange regions of the lung, for a given particle length the interception mechanism will become increasingly significant moving deeper into the lung. Accordingly, Chan and Gonda (1989) have previously noted that high aspect ratio drug particles are well suited for targeted delivery to the peripheral lung. Whereas these particles exhibit aerodynamic properties that reduce their deposition in the upper airways as compared to mass equivalent spherical particles, interception is expected to enhance their deposition in smaller, peripheral airways.

The strength of such an argument is, to a certain extent, limited by the tendency of elongated particles entrained in shear flow to align with their longer axes parallel to airway walls, thereby reducing the likelihood that they will deposit by interception. It has been predicted analytically that the tendency of solid, elongated particles is to rotate periodically, or tumble, in shear flow. These predictions have been validated experimentally for flow parameters within the range of those found in the lung. The angular velocity of the particle is not constant over a single rotation; rather, a maximum occurs when the particle's major axis is aligned perpendicular to the flow direction, and a minimum when this axis is parallel to the flow. As a result, in terms of orientation, the particle is predominantly aligned with its major axis parallel to the flow direction, and to airway walls.

### SUMMARY

Small airway deposition of orientable drug particles in the lung due to interception is increased through alignment of these particles with an externally applied force such as a magnetic field. Drug particles in one embodiment are made magnetically responsive by loading them with magnetic nanoparticles. Elongated particles have a natural tendency to align parallel to the direction of flow through an airway, and therefore also parallel to airway walls; accordingly, alignment with a magnetic field to any other orientation increases interception, with a maximum increase for alignment perpendicular to airway walls. By positioning a magnetic field across a specific site within the lung, for example in the area of a tumor, the increase in deposition by interception allows localized targeting of inhaled drug particles to that area. The elongated particles comprise a combination of orienting element and pharmaceutical active agent, and the orienting element is distributed in the particle to provide a torque on the particle in response to an external field. Localized targeting may be beneficial in the delivery of chemotherapeutic agents through inhalation. A moving magnetic field may be used to enhance deposition. These and other aspects of the particles, apparatus and method are set out in the claims, which are incorporated here by reference.

### BRIEF DESCRIPTION OF THE FIGURES

Embodiments will now be described with reference to the figures, in which like reference characters denote like elements, by way of example, and in which:

FIG. 1 is a schematic showing an apparatus for delivering orientable drug particles to the lung;

FIGS. 2A-2G show examples of orientable drug particles;

FIG. 3 is a schematic of the orientation of a prolate ellipsoid with respect to a linear shear flow F;

FIG. 4A is schematic of the magnetic field produced by an ideal dipole

FIG. 4B shows orientation of an external magnetic field, Hₒ, with a high aspect ratio drug particle's hard axis of magnetization, each dipole being opposed by the fields of its neighbors;

FIG. 4C shows orientation of Hₒ with a drug particle's easy axis of magnetization, where dipoles are stabilized by the fields of their neighbors;

FIG. 5 shows an apparatus for measuring the aerosol deposition in an array of small airway bifurcations (for particle size measurements, the airway array was removed and aerosol was sampled onto a polycarbonate membrane);

FIG. 6A shows a side view of semicircular cross-section, bifurcating channels cut into the side of a strip of aluminum;

FIG. 6B shows a top view of the airway array formed by stacking the aluminum strips;

FIG. 7 shows measured magnetic flux density along the centerline between the two permanent magnets spaced 13.5 cm apart, in which the shaded area represents the region where the magnetic field lines cross the airway;

FIG. 8 shows deposition efficiency in the small airway model for cromoglycic acid aerosol, and for magnetite-loaded cromoglycic acid aerosol with and without an aligning magnetic field, where error bars represent one standard deviation (n=3);

FIG. 9 shows the ratio between the magnetic torque and the aerodynamic torque for various particle aspect ratios and magnetite loadings (the aerodynamic torque is calculate from equations 2-4 with the flow velocity gradient G=8 U/3 R=1493 s⁻¹, U=14 cm/s is the average airway velocity and R=0.25 mm is the airway diameter in the present experiments.

FIG. 10 is a transmission electron microscope (TEM) image of cromoglycic acid (high aspect ratio particles (rods 36) loaded with magnetite (smaller, dark clusters).

### DETAILED DESCRIPTION

The term drug is used to describe any pharmaceutically active agent that may be used to treat an animal or human being.

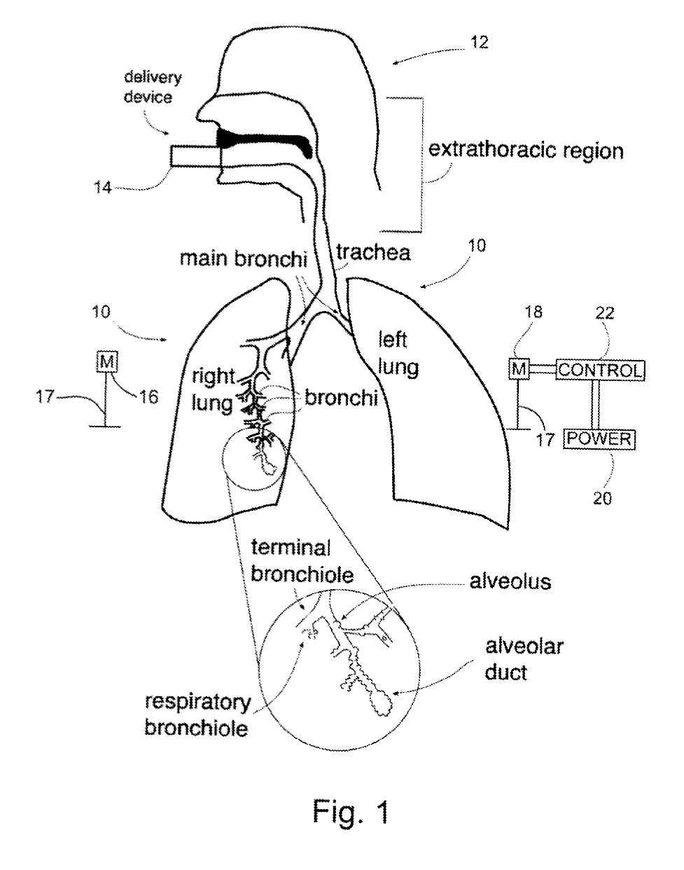

Referring to FIG. 1, there is shown an apparatus for targeted delivery of drug to the lung 10 of a human 12. The apparatus includes a drug delivery device 14 containing magnetically orientable drug particles and one or more magnetic field generators 16, 18. The drug delivery device 14 may be any device now know or hereafter developed that is capable of delivery of aerosol particles to the lung, as for example a nebulizer. The magnetic field generators 16, 18 may be for example one or more permanent magnets 16 or solenoids 18. In case of the use of solenoids 18, the solenoids may be activated by a power source 20 and controller 22. The controller 22 may be an on-off switch. The magnetic field generators 16, 18 are located over an area of the lung at which enhanced deposition is desired. Various methods may be used to fix the magnetic field generators at the correct location. For home application, the magnetic field generators may, for example, be held in pockets in a jacket. Or in a clinical application, the magnetic field generators may, for example, be part of a fixed apparatus in which the patient is placed.

In another embodiment, the magnetic field may be time varying or made to move, or spin or oscillate to enhance deposition. In such an embodiment, the multiple magnetic field generators 16, 18 may be spaced around the lungs of the patient and provided with time varying power from the controller 22 so as to create a time varying magnetic field, which may for example oscillate. Oscillation may for example be provided by alternating power to the magnetic field generators. A moving magnetic field may for example be provided by supplying power to a plurality of magnetic field generators out of phase with each other, so that the location of the magnetic field generator producing the strongest field moves around the patient. Hence, if there were three magnetic field generators 16, 18, electric energy having a sinusoidal pattern may be provided to the respective generators 120 degrees out of phase. Or two magnetic field generators 16, 18 may be 180 degrees out of phase for an oscillating field. The direction of oscillation may be in any direction chosen, in the plane of FIG. 1 or at any desired angle to the plane of FIG. 1. In one example, the polarity of the magnetic field may rotate around the patient, as for example using time varying fields or by using magnetic field generators 16, 18 that run on a track or tracks 17.

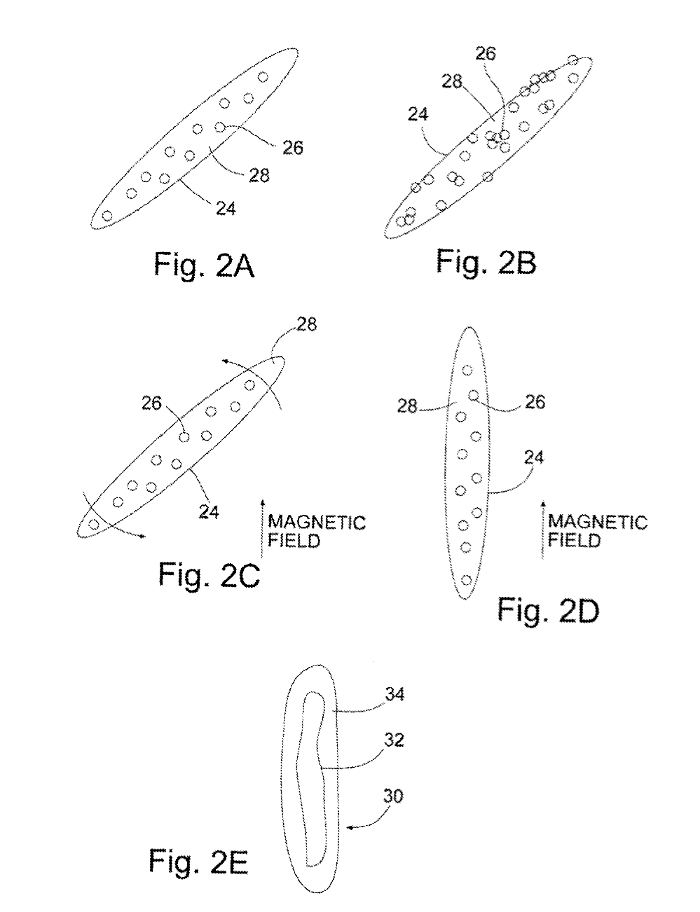

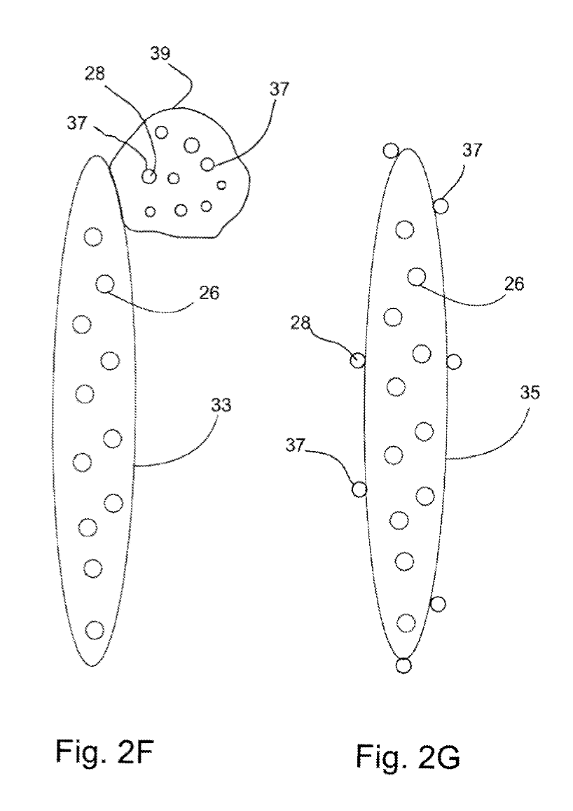

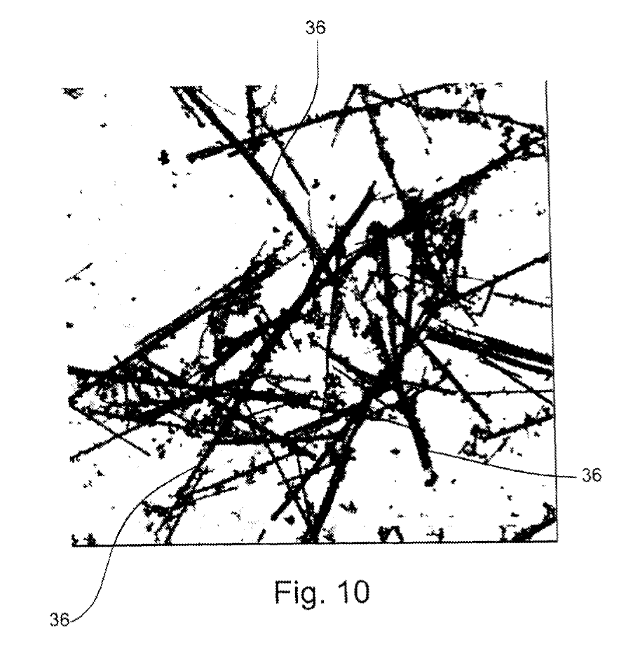

Exemplary orientable drug particles such as drug particles 24, 30, 35, 36 are shown in FIGS. 2A-2G and in FIG. 10. The description that follows will refer to drug particle 24, but applies in like manner to a drug particle 30, 33, 35, or 36. An orientable drug particle such as drug particle 24 comprises a combination of orienting element as for example orienting element 26 and drug 28. The orienting element 26 is distributed in the particle 24 to provide a torque on the particle 24 in response to an external field generated for example by the magnetic field generators 16, 18. In some embodiments, the orienting element 26 is a magnetically susceptible material such as iron. In order to provide torque on the particle 24 due to the magnetic field, the particle 24 has a length greater than its maximum width, as for example a length to width ratio greater than 3, or greater than 10 or 20. The orientable drug particles may be acicular particles 36 as shown in FIG. 10. Referring to FIGS. 2F-G, the drug 28 to be delivered may be contained in drug particles that are attached to the surface of the particles 33 and 35. Particles 33 and 35 may be elongated high aspect ratio particles 33 and 35. Referring to FIG. 2F, drug 28 is located in nanoparticles 37 that are contained within the matrix of at least one carrier particle 39. The carrier particles 39 may be designed to dissolve and release the nanoparticles 32, which deliver the active agent to the target cells. Referring to FIG. 2G, drug 28 may be located in nanoparticles 37 that are attached to particle 35. Nanoparticles 37 may be formed from, for example, the precipitation of a solution of a suitable polymer and drug 28. The surface of nanoparticles 37 may be modified by conventional methods to achieve, for example, a cell-specific uptake. The orientable drug particles in some embodiments may have a structure approximated by an ellipsoid or cylinder. In the case of the orientable drug particles having an approximately ellipsoid or cylindrical structure, the length of the particle is the length of the major axis of the ellipsoid or cylinder, respectively, and the maximum width is the maximum width of the ellipsoid or cylinder. As shown in FIG. 2E, an exemplary orientable drug particle 30 has a core 32 made of magnetically susceptible material such as iron and a drug coating 34, which may be a continuous or discontinuous coating of drug particles. Various methods may be used to make the drug particles 24, 30, 36 as for example the method described below.

The elongated particles 24, 30, 36 are especially adept at avoiding deposition in the upper airways, but exhibit enhanced deposition in the peripheral airways when oriented using the magnetic field. Particle sizes are preferred that limit deposition in non-targeted regions. Particles carried into non-targeted airways upon inhalation should have a low deposition efficiency, and be primarily removed upon exhalation, whereas the deposition efficiency of particles carried into targeted airways is increased through the external control.

Magnetic Vs. Aerodynamic Torque

Magnetic alignment of a high aspect ratio particle in transit through the lung requires that the magnetic torque exerted on the particle exceed the aerodynamic torque arising from shear in the entraining airflow. The latter torque for the low Reynolds number case of an ellipsoidal particle in a linear shear flow can be expressed as:

\vec{T}ₐₑ=μ└\vec{K}₁·(\vec{ω}_{f}−\vec{ω}ₚ)+\vec{K}₂·\vec{d}_{f}┘ (1)

where μ is the fluid viscosity, ω_{f} and ωₚ are the angular velocities of the fluid and the particle, the components of d_{f} relate to the shear strain of the fluid, and the components of tensors K₁ and K₂ depend only on the geometry of the particle, where these tensors can be diagonalized for a coordinate system corresponding to the principal axes of the particle.

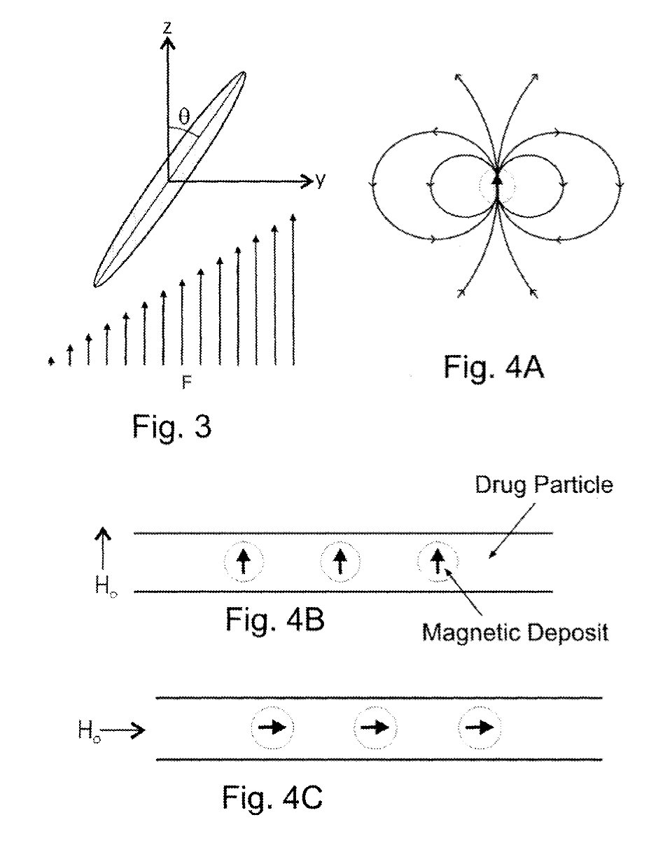

The aerodynamic torque on an elongated particle in the airways of the lung can be estimated from equation 1 if the shape of the particle is approximated as a prolate ellipsoid, and if the flow surrounding the particle is approximated as a linear shear. The second approximation is reasonable so long as the particle is small compared to the diameter of the airway. Considering a prolate ellipsoid fixed in space so that it cannot rotate, with its long axis in the velocity-gradient plane as depicted in FIG. 3, the component of the torque perpendicular to the plane is:

$${T}_{ae , x} = \frac{2 πμ {Gd}_{p}^{2} {l}_{p} ( {d}_{p}^{2} {cos}^{2} θ + {l}_{p}^{2} {sin}^{2} θ )}{3 ( {d}_{p}^{2} {β}_{o} + {l}_{p}^{2} {γ}_{o} )} \qquad ( 2 )$$

where G is the fluid velocity gradient, dₚ and lₚ are the diameter and length of the particle, the angle θ is defined in FIG. 3, and β₀ and γ₀ are:

$$\begin{matrix}{β}_{o} = \frac{{β}^{2}}{{β}^{2} - 1} + \frac{β}{2 {( {β}^{2} - 1 )}^{3 / 2}} ln ( \frac{β - \sqrt{{β}^{2} - 1}}{β + \sqrt{{β}^{2} - 1}} ) & ( 3 ) \\ and & \\ {γ}_{o} = \frac{- 2}{{β}^{2} - 1} - \frac{β}{{( {β}^{2} - 1 )}^{3 / 2}} ln ( \frac{β - \sqrt{{β}^{2} - 1}}{β + \sqrt{{β}^{2} - 1}} ) & ( 4 )\end{matrix}$$

where β is the particle aspect ratio, that is, the ratio between particle length and diameter.

For θ=90°, equations 2-4 can be used to estimate an upper limit for the aerodynamic torque exerted on an elongated particle aligned with an externally applied magnetic field. To maintain alignment, the magnetic torque on the particle should exceed this limit. As such, an estimate for the magnetic torque exerted on an elongated drug particle loaded with magnetic nanoparticles is highly desirable, though difficult to determine rigorously. A physical explanation for such a torque to exist can be made by considering neighboring deposits of magnetic material on a particle surface, as depicted schematically in FIG. 3. These deposits may be individual nanoparticles (FIG. 2a) or clusters of several nanoparticles (FIG. 2b), but here each will be approximated as an ideal magnetic dipole. FIG. 4a displays the magnetic field lines produced by a single dipole. In spherical coordinates, with origin at the center of the dipole, the dipole field is given by:

$${{H}^{->}}_{dip} = \frac{{MV}_{d}}{4 π {r}^{3}} ( 2 cos θ \hat{r} + sin θ \hat{θ} ) \qquad ( 5 )$$

where M is the magnetization of the deposited material, and V_{d} is the volume of the deposit.

FIGS. 4b and 4c show arrangements of neighboring dipoles for external magnetic fields oriented, respectively, perpendicular to and parallel to the long axis of the drug particle. For the perpendicular orientation, at any particular dipole, neighboring dipole fields act in the direction opposite to the dipole, whereas, for the parallel orientation, neighboring dipole fields act in the same direction as a given dipole. In other words, for the perpendicular orientation each dipole is destabilized by the fields of its neighbors, while in the parallel orientation each dipole is stabilized by the fields of its neighbors. For a large number of dipoles arranged over the surface of an elongated drug particle, the net result is that the composite particle exhibits anisotropic magnetization, with an easy axis of magnetization along the length of the particle, and will experience a magnetic torque tending to align its long axis with the external field.

In order to estimate the magnitude of this magnetic torque, consider first the arrangement of magnetic deposits depicted in FIGS. 4b and 4c, but with the external magnetic field oriented at some angle φ with respect to the particle's long axis. At the midpoint between any two dipoles, the vector sum of the fields produced by the two nearest dipoles can be calculated according to equation 5. Assuming that the dipoles are equally spaced, and that the corresponding deposits are of equal volume and spherical, the components of the net dipole field parallel and perpendicular to the particle's long axis are:

$$\begin{matrix}{H}_{dip , //} = \frac{4}{3} M cos {φ ( \frac{{d}_{d}}{L} )}^{3} & ( 6 ) \\ and & \\ {H}_{dip , \_ ∖ \_} = - \frac{2}{3} M sin {φ ( \frac{{d}_{d}}{L} )}^{3} & ( 7 )\end{matrix}$$

where d_{d} is the diameter of the deposit and L is the spacing between dipoles. As the field produced by a dipole decreases with the cube of the distance from the dipole (equation 5), including a larger number of dipoles in the derivation of equations 6 and 7 results in only small changes to their values.

The magnetic torque on the composite particle is given by:

$${{T}^{->}}_{m} = {μ}_{o} {\int }_{V}^{ } ( {{M}^{->}}_{p} \times {{H}^{->}}_{o} ) d V \qquad ( 8 )$$

where Mₚ is the magnetization of the particle, Hₒ is the external field strength, and μₒ is the permeability of free space.

As discussed above, the source of the torque on a non-magnetic particle (that is, a particle of very low magnetic susceptibility) loaded with smaller, magnetic particles is the magnetization anisotropy arising from interaction between neighboring magnetic deposits. In equation 8, this anisotropy should be accounted for in the magnetization of the composite particle; however, an established method for predicting this magnetization is not available. Instead, interpreting magnetization as the contribution to the total magnetic field inside matter produced by that matter (whereas the field strength H is the contribution from external sources), the magnetic torque can be estimated by replacing the magnetization in equation 8 with the dipole field given by equations 6 and 7. With this substitution, and for an external magnetic field oriented in the same plane as the particle's long axis, the magnetic torque acting on the particle in the direction normal to the plane is:

$${T}_{m} = 2 {μ}_{o} {VH}_{o} {M ( \frac{{d}_{d}}{L} )}^{3} sin φcos φ \qquad ( 9 )$$

where φ is the angle between the particle's long axis and the direction of the external magnetic field, and M is the magnetization of the deposited material, as in equations 6 and 7.

While significant approximations were made in deriving equation 9, it can be used in combination with equations 2-4 to make a coarse estimate of required magnetic loadings needed to overcome aerodynamic alignment of high aspect ratio particles.

### EXPERIMENTAL

### Preparation of Nebulizer Suspensions

### Example 1

In one example, high aspect ratio particles of cromoglycic acid (CA) were prepared by crystallization according to conventional methods (H. K Chan and I. Gonda, J. Aerosol Sci. 20, 157 (1989)). Dried CA powders were dispersed in deionized water to yield suspensions containing 2 mg CA/ml water. Superparamagnetic magnetite particles were prepared by precipitation following conventional methods (F. Y. Cheng, C. H. Su, Y. S. Yang, C. S. Yeh, C. Y. Tsai, C. L. Wu, M. T. Wu, and D. B. Shieh, Biomaterials. 26, 729 (2005)), that produce magnetite particles that are reasonably monodisperse in diameter, with a mean diameter of about 10 nm. Magnetite was added to the CA suspensions by one of two methods: in the first, dilute suspensions of magnetite in deionized water were sonicated for 30 minutes to deaggregate the magnetite particles as much as possible, then these suspensions were used to disperse the CA powders; in the second method, the magnetite was added in the last stage prior to crystallization of CA, and then the combined CA/magnetite powders were dispersed in deionized water. In either case, the concentration of magnetite in suspension ranged from 10 to 20% by weight over several preparations.

Formulations of CA, CA with magnetite added post-crystallization, and CA with magnetite added pre-crystallization, were nebulized using conventional jet nebulizers (Up-Draft II; Hudson Respiratory Care, Inc., Temecula, Calif.) driven by compressor (PulmoAide 5650C; DeVilbiss Canada, Barrie, ON), and drawn along with ambient, drying air at 10 l/min. into a large volume delivery chamber. Evaporation of the nebulized droplets left behind an aerosol of CA particles, and, for the two formulations containing magnetite, allowed any free magnetite particles not attached to CA particles in suspension to adhere to them upon drying of the droplet. The combined nebulization and drying process produced CA particles with diameters on the order of a few hundred nanometers, and lengths ranging from hundreds of nanometers to a few microns.

As a test of particle alignment with an external magnetic field, and of the effect of alignment on interception, aerosols produced from each of the three formulations were drawn from the delivery chamber through polycarbonate track-etched membrane filters with pore size of 5 μm and diameter of 47 mm (Isopore TMTP; Millipore, Billerica, Mass.). The membrane filters were sealed tightly within an in-house filter casing, and the flow rate through the membrane was monitored to within 0.21±0.02 l/min. with a low flow rate rotameter (FL-2010; OMEGA Canada, Laval, QC). A bacterial air filter (Respirgard; Vital Signs, Inc., Totowa, N.J.) was placed downstream from the membrane to capture the aerosol that penetrated the membrane. The masses of CA collected on the membrane and the bacterial filter were measured by washing with 0.01 N sodium hydroxide, to convert the CA to its sodium salt, and subsequent assay by UV spectrophotometry (8452A; Hewlett-Packard, Palo Alto, Calif.) at a wavelength of 326 nm. On average, the total mass recovered from both filters (i.e. the challenge mass) was 72±16 μg (mean± one standard deviation, n=12).

Penetration efficiency, calculated as the mass of CA recovered from the bacterial filter as a percentage of the total mass recovered from both filters, was determined for each formulation, with and without the presence of a magnetic field across the membrane filter. The magnetic field was produced parallel to the face of the membrane by placing neodymium permanent magnets (2″×2″×0.5″ N42; Indigo Instruments, Waterloo, ON) on either side of the filter casing. The flux density at the center of the membrane was measured using a gauss meter (F.W. Bell 5180; Sypris Test and Measurement, Orlando, Fla.) to be 90 mT.

Measurement of the penetration efficiency for each formulation, with and without the magnetic field across the face of the membrane filter, showed that for the formulation of CA alone, there was no difference in the penetration of aerosol particles through the membrane with and without the magnetic field. In contrast, for both the formulations containing magnetite, penetration efficiency decreased significantly (one tailed student's t-test, p<0.05) when the magnetic field was produced across the face of the membrane. Neither of the two methods for formulating CA and magnetite proved clearly superior to the other; however, from a handling perspective, adding the magnetite prior to crystallization of CA creates as an end product a powder that needs only to be dispersed in water prior to nebulization.

### Example 2

The preparation of nebulizer suspensions containing high aspect ratio cromoglycic acid (CA) particles in superparamagnetic magnetite followed the procedure of example 1. Prior to each nebulization, 6 mg of dried CA/magnetite powder was dispersed in 3 ml of deionized water. The concentration of magnetite in suspension ranged from 10% to 20% by weight over several preparations. For comparison, suspensions containing 2 mg/ml of CA alone were also prepared and nebulized.

### Nebulization Efficiency

CA and CA/magnetite suspensions were aerosolized using Hudson Updraft II jet nebulizers (Hudson Respiratory Care, Inc., Temecula, Calif.) driven by a PulmoAide compressor (5650C; DeVilbiss Canada, Barrie, ON). Nebulization efficiency, defined here as the percentage of cromoglycic acid escaping the nebulizer, was determined for 3 ml nebules. A T-piece was attached to the nebulizer, and an absolute filter (Respirgard; Vital Signs, Inc., Totowa, N.J.) was placed at one end. A continuous flow of 10 l/min. was maintained by vacuum pump through the T-piece towards the filter, ensuring that no aerosol escaped through the opposite end. Nebulizers were run until their output became intermittent, and the same three nebulizers were tested for each formulation. The masses of CA captured on the filter, and remaining in the nebulizer and T piece, were determined by washing with 0.01 N sodium hydroxide, to convert the CA to its sodium salt, and subsequent assay by UV spectrophotometry (8452A; Hewlett-Packard, Palo Alto, Calif.) at a wavelength of 326 nm.

### Aerosol Particle Size and Concentration

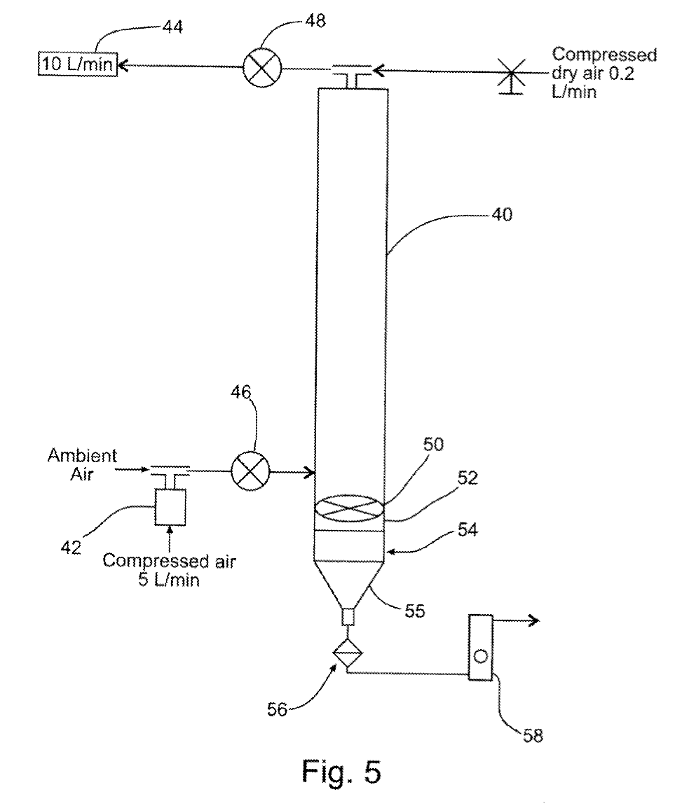

The particle size distribution and number concentration of the CA and CA/magnetite aerosols were determined from samples taken from the delivery apparatus used for the aerosol deposition experiments. Suspensions of CA and CA/magnetite were nebulized, and drawn by vacuum pump along with ambient, drying air at 10 l/min. into a large volume (˜16 1) delivery chamber for 1.5 minutes. With reference to FIG. 5, after filling the delivery chamber 40 with aerosol, valves 46, 48 connecting to the nebulizer 42 and the vacuum pump 44, respectively, were closed, and a butterfly valve 50 at the base 52 of the chamber 40 was opened. Aerosol was then sampled from the base 52 of the chamber 40 onto a 0.2 μm pore polycarbonate membrane (Isopore GTTP04700; Millipore, Billerica, Mass., not shown) for 1 hour at a flow rate of 0.21 l/min., maintained through the delivery chamber using dry, compressed air and a needle valve, and monitored using a low flow rate rotameter (FL-2010; OMEGA Canada, Laval, QC). Over the period of the sampling, the needle valve was adjusted as required to hold the flow rate to within 0.21±0.02 l/min.

After collecting aerosol from the delivery chamber, samples of the polycarbonate membranes were prepared for analysis by scanning electron microscopy (SEM) (S-2500; Hitachi, Japan). For both the CA and CA/magnetite aerosols, three pieces were cut from the membrane, from locations chosen at random, and mounted to SEM stubs using two-sided carbon adhesive tabs. These samples were sputter coated with a thin layer of gold prior to SEM analysis. Images of the samples were taken at 3000× magnification, again from locations chosen at random, and stored digitally.

For three SEM images from each of the three membrane samples, the length and diameter of each particle was manually measured using digital image analysis software (Scion Image; Scion Corporation, Frederick, Md.). The particles were assumed to lie parallel to the face of the membrane. Particle sizing rules were similar to those established for asbestos samples by Platek et al. (1992). Each particle was marked immediately after being sized so as to avoid sizing the same particle twice. Where a particle was not perfectly straight, its length was measured as the arc length along its central axis from one tip to the other. Diameter was measured perpendicular to the central axis at the midpoint along a particle's length, except in rare cases where the particle had two regions of clearly different diameter. In these latter cases, an average of the two diameters was taken. Overlapping particles were sized separately only if both ends of each particle were clearly visible.

After sizing, the volume of each particle was calculated, under the approximation that the particles were cylindrical. Volume-weighted size distributions were then fit with lognormal curves by nonlinear regression to yield a volume median length (VML), volume median diameter (VMD), and geometric standard deviations in both length and diameter (σ_{L} and σ_{D}), respectively) for each SEM image. In addition, the particle number concentration in the sampled air was calculated for each image from the volume of sampled air, and the ratio between the image area and the total area of the membrane exposed to the aerosol. The mean values of these parameters for the three different membrane samples were compared by one-way ANOVA for independent samples in order to gauge their variation over different locations on the membrane.

### Generation of Magnetic Field

The magnetic field used in the airway deposition measurements was generated using neodymium permanent magnets (2″×2″×0.5″ N42; Indigo Instruments, Waterloo, ON). Pairs of 0.5″ thickness magnets were stacked to form two 1″ thickness magnets. These two magnets were positioned 13.5 cm apart, with opposite poles facing one another. The magnetic flux density was measured along the center axis between the two magnets using a gauss meter (F.W. Bell 5180; Sypris Test and Measurement, Orlando, Fla.).

### Design of Small Airway Array

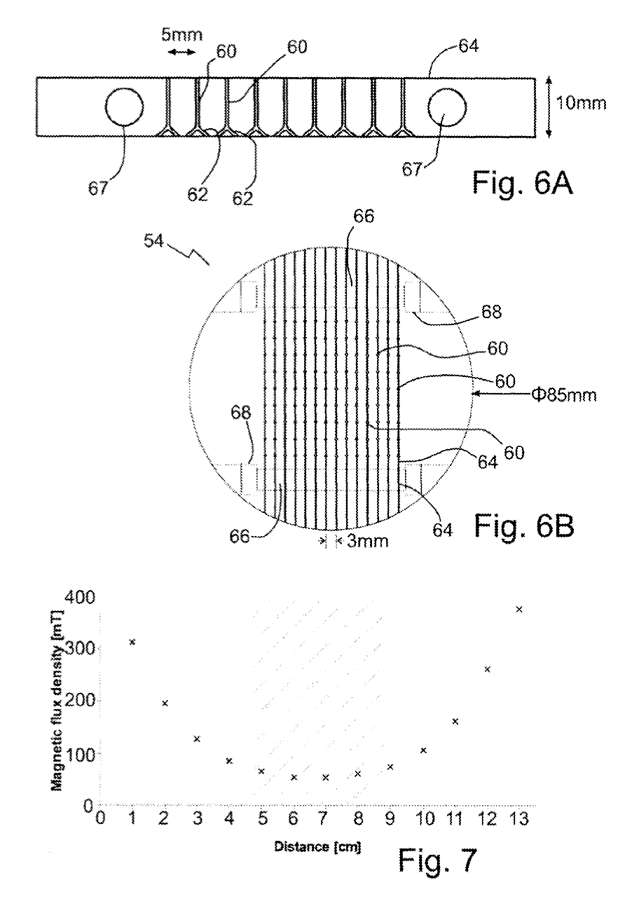

Aerosol deposition experiments were performed in an array 54 of small, bifurcating airways as shown in FIG. 6a, 6b. As designed, parent airway 60 and daughter airway 62 diameters were 0.5 mm, and the branching angle of each daughter airway was 50°. The length of the parent airways 60 was 8 mm, and that of the daughter airways 62 was 2 mm. The radius of curvature between the parent and daughter airways was 1 mm, while that of the carinal ridge was 0.05 mm. These airway dimensions were chosen to be representative of those found in the terminal bronchioles of the human lung, with the exception of the parent to daughter diameter ratio, and the length of the parent airway. A parent to daughter diameter ratio of 1 is somewhat lower than is anatomically realistic; however, an equal diameter for the parent and daughter airways simplified machining of the airways considerably. Likewise, the long parent airways were required in order to maintain the dimensions of the model above a minimum workable size.

To build the airway model, a row of nine semicircular cross-section, bifurcating channels were cut into both sides of thirteen 3 mm thick strips of aluminum, and into one side of each of two thicker end pieces, using a CNC milling maching. As seen in FIG. 6a, on a given strip 64, each parent airway 60 was separated by 5 mm from its neighbors on either side. Referring to FIG. 6b, the strips 64 of aluminum were stacked together to form a nine by fourteen array 54 of circular cross-section bifurcations, depicted schematically. Brass dowels 66 were used to align the stacked strips 64. Referring to FIG. 6A, holes 67 are provided in each strip 64 for passage of the dowels 66 (shown in FIG. 6b) therethrough. The surfaces of the strips 64 were lapped, and screws 68 at either end of the dowels 66 held the stack together to ensure an airtight seal between neighboring surfaces.

After machining the channels, their dimensions were measured by analysis of digital images taken through a stereomicroscope coupled with a digital camera (DXM 1200; Nikon, Japan) at 100× magnification. Within the resolution of the measurements (±0.01 mm for lengths and ±2° for angles) neither the airway lengths nor the branching angles varied from the design parameters. Measurements of channel widths and radii of curvature were taken for two bifurcations from each of five strips chosen at random. The average parent airway width was 0.53±0.03 mm (mean± one standard deviation, n=10), while the average daughter airway width was 0.51±0.02 mm (n=20). The average radius of curvature between parent and daughter airways was 0.79±0.07 mm (n=20). Carinal ridges were observed to be very sharp, to an extent that their radii of curvature could not be accurately measured. The depths of the channels were measured by stacking and securing the strips to form the airway model, and then, from a top view of the model, measuring the diameter of airways formed between channels of opposing strips in the direction perpendicular to the faces of the strips. The average airway diameter measured in this manner was 0.52±0.04 mm (n=36).

### Small Airway Deposition

Referring to FIG. 5, the experimental apparatus used to measure aerosol deposition in the airway array was identical to that from which aerosol was sampled for particle size measurements, except that the airway array 54 was placed in an aluminum holder 55 positioned at the base 52 of the delivery chamber 40, as depicted. In this position, the parent airways were oriented parallel to the direction of gravity. An absolute filter 56 (Respirgard; Vital Signs, Inc., Totowa, N.J.) was placed downstream from the array holder 55 to collect aerosol particles that escaped deposition in the airways. For each experimental run, a CA or CA/magnetite suspension was nebulized, and drawn with additional drying air into the delivery chamber 40 at 10 l/min. for 1.5 minutes. Next, valves 46, 48 connecting to the nebulizer 42 and the vacuum pump 44 were closed, and the butterfly valve 50 at the base 52 of the chamber 40 was opened. Compressed air was then used to maintain a continuous flow of air from the delivery chamber 40 through the airway array 54 for 1 hour, at a flow rate of 0.21±0.02 l/min. As in the particle sizing procedure, the flow rate was monitored using a rotameter 58. Because the deposition efficiency in the airway array was low, this cycle of filling the delivery chamber and then draining it through the array was repeated twelve times for each experimental run in order to allow a measurable mass of CA to deposit in the array. Three experimental runs were performed for both CA and CA/magnetite without the magnetic field in place, and another three runs were performed for CA/magnetite with the magnetic field aligned across the array, that is, perpendicular to the parent airways.

The masses of CA deposited in the array 54, the array holder 55, and the downstream filter 56 were determined by washing with 0.01 N sodium hydroxide, to convert the CA to its sodium salt, and subsequent assay by UV spectrophotometry at a wavelength of 326 nm. The deposition efficiency in the array was calculated as the mass of CA deposited in the array as a percentage of the total mass of CA recovered from the array, the holder, and the downstream filter. Only the section of the holder downstream from the array was washed. As the top surface of the array was exposed to the delivery chamber during the experiments, prior to washing the array any aerosol that had settled onto the surface was removed. This was accomplished first using masking tape to repeatedly lift aerosol off the surface, and then by cleaning the surface with cotton swabs dipped in 0.01 N sodium hydroxide.

### Results

### Nebulization Efficiency

The measured nebulization efficiencies and run times for CA suspension with and without added magnetite are listed in table 1. Clearly, for the three Updraft II nebulizers tested, the addition of superparamagnetic magnetite particles to the suspension did not significantly alter the nebulization efficiency or run time. For the CA suspensions, the nebulization efficiency was 43.3±13.4%, and the run time was 7.39±0.58 minutes, while for the CA/magnetite suspensions, the nebulization efficiency was 40.5±15.2%, and the run time was 7.07±0.56 minutes. With reference to table 1, it appears that the large standard deviation between runs was due to variation between nebulizers, as opposed to experimental error.

### Particle Size and Concentration

Lognormal length and diameter distributions, and particle number concentrations, were determined for both the CA and CA/magnetite aerosols from SEM images of three samples taken from different locations on the sampling membranes. In order to evaluate the heterogeneity of collected aerosol across the membrane, one-way ANOVA for independent samples was used to compare the VMD, VML, σ_{L}, σ_{D}), and the estimated number concentration (N) across the three membrane samples for each formulation. For CA, no statistically significant differences were found between membrane samples for any of these parameters (P>0.05). For the CA/magnetite, statistically significant (P<0.05) differences in VMD and σ_{D} were found between two of the samples, and in N between one sample and the other two, while there were no significant differences between samples for the length distribution parameters. VMD ranged from 0.43±0.04 μm to 0.52±0.02 μm between samples, while σ_{D}) ranged from 1.47±0.08 to 1.68±0.02 and N ranged from 22480±1390 cm⁻³ to 29919±2834 cm⁻³. As these differences, though statistically significant, are reasonably small, for both formulations the mean and standard deviation of each parameter was calculated over values from all nine SEM images taken from the three different membrane samples. These are reported in table 2.

### Magnetic Field

The magnetic field generated between the two permanent magnets was measured along the centerline between the two magnets using a gauss meter. FIG. 7 displays the measured magnetic flux densities between the magnets, and indicates the region where the magnetic field lines crossed the airways. Within this region, the measured flux density dropped from ˜75 mT at the edges to ˜55 mT in the center.

### Small Airway Deposition

The aerosol deposition efficiency in the airway array was calculated as the mass of CA recovered from the array as a percentage of the total mass of CA recovered from the array, the holder, and the downstream filter. The total recovered mass of CA averaged over all experiments was 1.5±0.3 mg (n=9). There was no significant difference in the total mass recovered between experiments performed for CA, and for CA/magnetite with and without the magnetic field (one way ANOVA, standard weighted means analysis for independent samples; F=0.57).

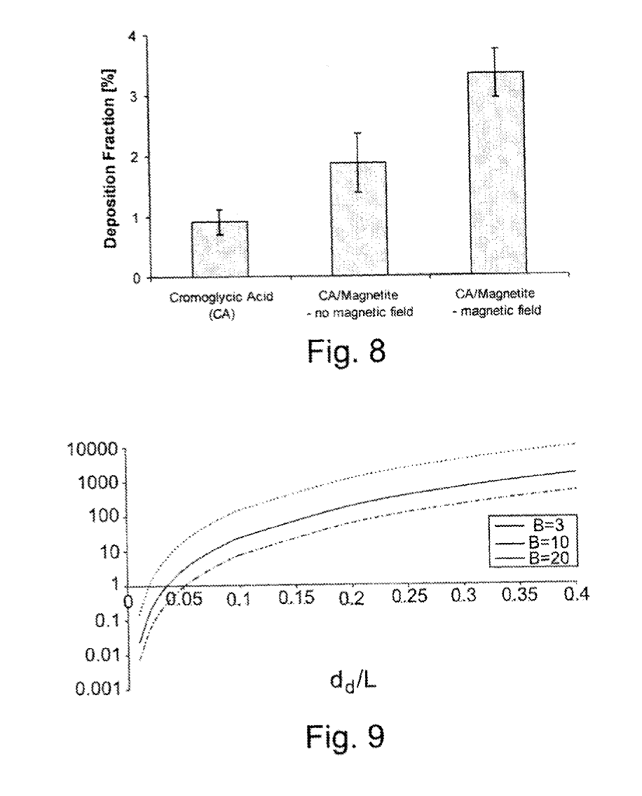

The deposition efficiencies for the three cases studied are shown in FIG. 8. The deposition efficiency for CA was 0.9±0.2%, while for CA/magnetite it was 1.9±0.5% with no magnetic field, and increased to 3.3±0.4% with the magnetic field positioned across the array. Employing one way ANOVA, with standard weighted means analysis for independent samples, and the Tukey HSD test, the difference between deposition efficiencies for CA and for CA/magnetite with no magnetic field lies just outside the range of statistical significance (P>0.05), whereas the increased deposition efficiency observed for CA/magnetite in the presence of the external magnetic field is statistically significant (P<0.01).

Deposition of magnetite-loaded, high aspect ratio drug particles in small, bifurcating airways is increased when a magnetic field is produced across the airways. Airway dimensions were chosen to be on the same order as those of the terminal bronchioles in the human lung. Airway diameters are sufficiently small that interception plays a major role in determining the deposition of particles a few micrometers in length. While airway diameters are smaller still in more peripheral, gas-exchange regions of the lungs, these airways are heavily lined with alveoli, and undergo significant expansion and contraction over a breathing cycle, making the design of an anatomically accurate physical model extremely difficult.

In one model of the lung, the terminal bronchioles occur at the 14^{th} generation of the lung, where the generation number of a particular airway refers to the number of branches separating that airway from the trachea. Assuming that the bifurcations in the airway array used in the present study represent branching from the 14^{th} to the 15^{th} lung generation, and that that the flow through the array divides evenly into the 126 parent airways, the flow rate of 0.21 l/min. through the array corresponds to an inhalation flow rate of 27.3 l/min. at the trachea. Alternatively, in a diameter-based reconstruction of the conducting airways, wherein the average velocity through a 0.5 mm diameter airway is approximately 10% of the average velocity through the trachea, assuming a tracheal diameter of 1.8 cm, the flow rate through the array corresponds to a somewhat lower inhalation flow rate of 21.6 l/min. at the trachea.

The addition of magnetite to suspensions of CA had no impact on the nebulization efficiency of CA from Updraft II jet nebulizers. However, the VMD and VML of the aerosol sampled from the delivery chamber were both larger for the combined CA/magnetite formulation than for CA alone. This increase in particle size may be due to increased aggregation of CA particles in the presence of colloidal magnetite, and likely explains the increased deposition efficiency of CA/magnetite in the airway array compared to CA alone.

Deposition of magnetite-loaded CA aerosols in the airway array increased by 74% with a 55 mT magnetic field aligned perpendicular to the parent airways, as compared to deposition with no magnetic field. This outcome demonstrates the feasibility of magnetic alignment of high aspect ratio particles as a means to achieve localized targeting of inhaled aerosols in the peripheral airways. In order to improve open this initial result, that is, to achieve a larger increase in deposition, a first question to be addressed is the extent to which particles were aligned with the magnetic field in the present experiment. An estimate can be made for the ratio between the magnetic torque and the aerodynamic torque acting on the particles within the airways using equations 2-4 and equation 9. FIG. 9 displays the relationship between this ratio and the amount of magnetite loading for different particle aspect ratios. Even for an aspect ratio of 20, which is at the upper extreme of the CA/magnetite particles sized in the present study, the magnetic torque is expected to be much greater than the aerodynamic torque (Tm/Tae >10) for deposits of magnetite spaced up to 9 diameters apart. FIG. 10 displays a transmission electron microscopy (TEM) image of the CA/magnetite formulation dried from suspension. Although the level of magnetite loading varies considerably between CA particles, the vast majority of particles appear to contain sufficient loadings to overcome the aerodynamic torque, and achieve magnetic field alignment. As such, increases to the magnetic field strength, or to the concentration of magnetite in suspension, are not expected to increase deposition above that measured in the present experiment. Instead, optimization of particle morphology is likely to be the avenue through which deposition in targeted areas is further increased. Longer particles will deposit more readily by interception when aligned in targeted regions, whereas their deposition by impaction or sedimentation in non targeted areas will change very little, owing to the weak dependence of aerodynamic diameter on the length of high aspect ratio particles.

Unlike previously proposed techniques for magnetically targeted drug delivery dating back now three decades, the present technique does not rely on a magnetic force on particles, but rather on a magnetic torque. Accordingly, no gradient in the magnetic field is required to target particles, eliminating an obstacle typically associated with techniques that rely on a magnetic force. As seen in FIG. 7, in the present work the magnetic field was relatively constant across the airways, with a gradient of less than 1 T/m through most of the array. Increasing the field gradient to larger values would likely result in a greater increase to deposition in the airway array, due to drift of particles towards airway walls resulting from a magnetic force; however, it is uncertain whether such a result could translate to clinical applications in humans because high field gradients are difficult to generate at sufficient depth below the surface of the skin.

The optimal length of the particle will depend on diameter of the airway (or airways) to be targeted. In the human lung, airway diameters decrease from approximately 1.8 cm at the trachea down to approximately 300 micrometers in the alveolar (deepest) airways. A possible upper limit for the particle length in any given application is the diameter of the airway(s) to be targeted. To target the peripheral lung, this means an upper limit of about 500 μm. However, the technology may be used to target larger airways closer to the trachea, and in that case much longer particles may be used. A good lower limit for the particle length would be 1 micrometer, as this is much smaller than any airway diameter in the lung. Particle width is selected to keep the deposition due to inertial impaction and gravitational sedimentation minimal in non-targeted airways. The particle properties that affect these deposition mechanisms are size, shape, and density. It is customary in aerosol science to summarize these properties in an equivalent aerodynamic particle diameter, which is the diameter of a sphere with density =1 gram/cubic centimeter that has the same aerodynamic properties as the particle in question. Particles with aerodynamic diameter 10 micrometers are generally impractical for delivery of drugs to the lung since most of these particles are filtered out by the mouth and throat. Therefore, an absolute upper limit on aerodynamic diameter may in some embodiments be about 10-15 micrometers. For high aspect ratio particles, aerodynamic diameter depends almost linearly on particle width, and only weakly on particle length, so that an upper limit on width of 10-15 micrometer is probably reasonable for most particles (i.e. all but those with very low density) to be inhaled. For the particular application of targeting to certain peripheral airways, widths smaller than about 1-3 micrometers would be preferable.

In the claims, the word “comprising” is used in its inclusive sense and does not exclude other elements being present. The indefinite article “a” before a claim feature does not exclude more than one of the feature being present. Each one of the individual features described here may be used in one or more embodiments and is not, by virtue only of being described here, to be construed as essential to all embodiments as defined by the claims. Immaterial modifications may be made to the embodiments described here without departing from what is covered by the claims.

TABLE 1
Nebulization Efficiencies and Run Times for
Cromoglycic Acid Suspensions with and without Magnetite

| | | | Cromoglycic Acid | |
|---|---|---|---|---|
| | Cromoglycic Acid | | with Magnetite | |
| Nebulizer | Efficiency (%) | Time (min.) | Efficiency (%) | Time (min.) |
| A | 49.3 | 6.95 | 49.4 | 6.87 |
| B | 52.6 | 7.17 | 49.2 | 6.63 |
| C | 27.9 | 8.05 | 23.0 | 7.70 |
| Average | 43.3 ± 13.4 | 7.39 ± 0.58 | 40.5 ± 15.2 | 7.07 ± 0.56 |

Average values expressed as mean ± one standard deviation,
n = 3.

TABLE 2
Particle Size Distributions and Number
Concentrations for Cromoglycic Acid and Magnetite-Loaded
Cromoglycic Acid Aerosols

| | CA/magnetite | CA |
|---|---|---|
| VMD [μm] | 0.47 ± 0.05 | 0.34 ± 0.02 |
| σ_{D} | 1.6 ± 0.1 | 1.5 ± 0.1 |
| VML [μm] | 3.0 ± 0.5 | 2.0 ± 0.4 |
| σ_{L} | 2.1 ± 0.2 | 2.1 ± 0.1 |
| N [cm⁻³] | (2.5 ± 0.4) × 10⁴ | (2.0 ± 0.3) × 10⁴ |

Values are expressed as mean ± one standard deviation,
n = 9.
VMD, VML are the volume median diameter and length, respectively.
σ_{D} and σ_{L} are the geometric standard deviations for diameter and length, respectively.
N is the number of particles per unit volume of sampled air.

What is claimed is:

1. A particle comprising a combination of orienting element and drug, and the orienting element is distributed in the particle to provide a torque on the particle in response to an external field.

2. The particle of claim 1 in which the orienting element is a magnetically susceptible material.

3. The particle of claim 1 in which the particle has a length greater than its maximum width.

4. The particle of claim 3 in which the particle has a length to width ratio greater than 3.

5. The particle of claim 1 in which the particle is acicular.

6. The particle of claim 2 in which the drug is contained in a drug particle attached to a surface of the particle.

7. A method for delivery of a drug to a lung, the method comprising the steps of:
directing particles comprising a drug into passageways of the lung; and
altering the orientation of the particles when the particles arrive at a selected location in the lung.

8. The method of claim 7 in which the particles comprise particles of claim 1.

9. The method of claim 7 in which the orientation of the particles is altered by a magnetic field external to the lung.

10. The method of claim 9 in which the magnetic field is generated by a solenoid and turned on at a chosen time.

11. The method of claim 9 in which the magnetic field is a time varying magnetic field.

12. The method of claim 11 in which the magnetic field is an oscillating field.

13. The method of claim 11 in which the magnetic field is a rotating magnetic field.

14. Apparatus for targeted delivery of drug to the lung, the apparatus comprising:
a drug delivery device containing magnetically orientable drug particles; and
a magnetic field generator.

15. The apparatus of claim 14 in which the magnetic field generator is configured to provide a time varying field.

16. The apparatus of claim 15 in which the magnetic field generator is configured to provide an oscillating or rotating magnetic field.
